(19) United States
(12) Patent Application Publication
(10) Pub. No.: US 2023/0038720 A1
(43) Pub. Date: Feb. 9, 2023

(54) Compositions and Methods for Treating Multi-Resistant Bacterial Infection

(71) Applicant: The University of Southern California, Los Angeles, CA (US)

(72) Inventors: Michael E. Selsted, Pasadena, CA (US); Dat Q. Tran, Alhambra, CA (US); Justin B. Schaal, Orange, CA (US)

(21) Appl. No.: 17/948,904

(22) Filed: Sep. 20, 2022

Related U.S. Application Data
(63) Continuation-in-part of application No. 17/314,473, filed on May 7, 2021.
Application No. 17/314,473 is a continuation of application No. 16/914,038, filed on Jun. 26, 2020, now Pat. No. 11,021,518.
(60) Provisional application No. 63/246,176, filed on Sep. 20, 2021.
(60) Provisional application No. 62/867,000, filed on Jun. 26, 2019.

(51) Int. Cl. C07K 7/64; A61P 31/00

(52) U.S. Cl. CPC C07K 7/64; A61P 31/00; A61K 38/00

(57) ABSTRACT

Peptide analogs of θ-defensins have been developed that are effective in treating multi-drug resistant microbial infections when used at concentrations below those needed to provide a microbicidal effect. These peptides were found to modulate host defense mechanisms to increase clearance of microbial pathogens, enhance phagocytic activity, and enhance neutrophil recruitment while reducing inflammation.

This application claims the benefit of U.S. Provisional Patent Application No. 63/246,176, filed Sep. 20, 2021, and is a continuation in part of U.S. patent application Ser. No. 17/314,473, filed May 7, 2021, which is a continuation of U.S. Pat. No. 11,021,518, filed Jun. 26, 2020, which claims the benefit of U.S. Provisional Patent Application No. 62/867,000 filed on Jun. 26, 2019. This and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

This invention was made with government support under Grant Nos. R01 AI125141 and R44 AR068833, awarded by the National Institutes of Health (NIH). The government has certain rights in the invention.

### FIELD

The field is treatment of infection by multi-drug resistant bacteria.

### BACKGROUND

The background description includes information that may be useful in understanding the present disclosure. It is not an admission that any of the information provided herein is prior art or relevant to the presently claimed invention, or that any publication specifically or implicitly referenced is prior art.

The emergence of infections by carbapenem resistant Enterobacteriaceae (CRE) represent a global threat to human health. The Centers for Disease Control and Prevention (CDC) and the World Health Organization (WHO) categorize CRE infections as a major and urgent threat to public health. Resistance of CRE to carbapenem antibiotics leaves few treatment options other than colistin and polymyxin B, both of which have limited use because of their toxicities. Among Enterobacteriaceae, infections by Klebsiella pneumoniae and Escherichia coli cause an estimated 140,000 nosocomial infections per year in the United States alone, and an increasing fraction are carbapenem resistant. Of the β-lactam antibiotics, carbapenems have the broadest activity spectrum and greatest potency against Gram negative bacteria. However, the incidence of CRE infections continues to rise in health care settings and in the community^{4,5}, underscoring the need for new therapeutic countermeasures. In this regard, most efforts have focused on developing new carbapenem/β-lactam-based drugs, some which are combined with β-lactamase inhibitors³. Carbapenem resistance is multifactorial, involving acquisition of new or mutant β-lactamases, efflux pumps, loss of outer membrane porins, and alterations of penicillin binding proteins^{6,7}, and combinations of these resistance mechanisms broaden resistance to carbapenems and other antibiotics by CRE 5, especially in K. pneumoniae.

Defensins are a diverse family of small antimicrobial proteins that are part of the body's nonspecific defense against infection. There are three different and structurally distinct classes of defensin proteins: alpha, beta, and theta defensins. The α and β defensins are linear, tri-disulfide containing peptides having molecular weights of about 2.6 kDa or 4.5 kDa, respectively. In contrast, θ-defensins are cyclic peptides (i.e. circular peptides wherein the backbone is formed by sequential peptide bonds with neither a free amino or carboxyl terminus) composed of 18 amino acids.

θ-defensins are expressed in tissues of rhesus monkeys, baboons, and other Old World monkeys. They are not present in humans and other hominids. Naturally occurring θ-defensins are composed of 18 backbone cyclized (i.e. through the alpha-amine groups rather than side chain moieties) peptides stabilized by three disulfide bonds. These three disulfide bonds are conserved among all known θ-defensins. θ-defensins were originally discovered and classified as defensins based on the antimicrobial properties of the peptides. More recently it has been found that θ-defensins can have potent immunomodulatory effects.

International Patent Application Publication No. WO 2007/044998 (to Leherer et al) describes relationships between structure and biological activity for retrocyclin peptides and analogs of such peptides that include varying degrees enantiomer content in an attempt to derive structure/activity relationships. These analogs, however, retain the length and structure of the native retrocyclin. In addition, the reference is only instructive for antibacterial activity.

Peptide analogs of various defensins have been investigated. For example, European Patent Application EP2990415 (to Colavita et al) describes circularized analogs of a β-defensin that show improved antibiotic effectiveness relative to the parent protein. Such β-defensins, however, have been shown to stimulate release of pro-inflammatory cytokines, which raises safety concerns and limits their utility.

United States Patent Application Publication No. US 2003/0022829 (to Maury et al) describes synthesis and biologic activity of chimeric θ-defensins and speculates on the possibility of making conservative amino acid substitutions, however these appear to retain the length and structure of native θ-defensins.

Thus, there is still a need for safe and effective compounds for the treatment of multidrug resistant pathogens.

### SUMMARY

The inventive subject matter provides θ-defensins and synthetic analogs of θ-defensins that are useful in treating multi-drug resistant microbial infections. These peptides are effective at concentrations that are do not have direct microbicidal and/or microbistatic effect, and modulate a range of host defense mechanisms while suppressing inflammation.

Embodiments of the inventive concept include methods of treating an infection with a microbe in a subject in need thereof (which can be resistant to one or more antibiotics), by administering a small peptide in an amount effective to modulate at least two or at least three of microbial clearance, phagocytosis, neutrophil recruitment, and septic shock, using an amount does not provide a direct antimicrobial effect. Suitable small peptides include macrocyclic peptides, θ-defensins, and/or θ-defensins analogs. Such modulations can include enhancing phagocytosis of the microbe, enhancing clearance of the microbe, increasing host neutrophil recruitment, reducing or increasing concentration of a pro-inflammatory cytokine, and/or reducing or increasing concentration of an anti-inflammatory cytokine. In some embodiments the small peptide is selected to be resistant to a microbial protease.

Small peptides suitable for such methods include cyclic peptides can have a structure as shown in FIG. 2A, where AA3 and AA12 are cysteines joined by a disulfide bond, AA5 and AA10 are cysteines joined by a disulfide bond, AA4 is a first hydrophobic amino acid, AA11 is a second hydrophobic acid, AA6 is arginine, AA7 is arginine, AA8 is arginine, and wherein the cyclic peptide has four arginine residues that provide a positively charged content of about 28% at physiological pH. Alternatively, suitable small peptides can include cyclic peptides that have a structure as shown in FIG. 2A, where AA3 and AA12 are cysteines joined by a disulfide bond, AA5 and AA10 are cysteines joined by a disulfide bond, AA4 is serine or a first hydrophobic amino acid, AA11 is serine or a second hydrophobic acid, AA6 is arginine, AA7 is arginine, AA8 is arginine, and wherein the cyclic peptide comprises five arginine residues that provide a positively charged content of at least about 36% at physiological pH.

Another embodiment of the inventive concept is a composition for treating microbial infection in a subject in need thereof (for example, infection with a drug resistant or multi-drug resistant microbe) that includes a small peptide selected to be effective in modulating at least two or at least three of microbial clearance, phagocytosis, neutrophil recruitment, and septic shock in an amount does not provide a direct bactericidal effect, as well as an antibacterial antibiotic, an antiviral, an antifungal antibiotic, an anti-inflammatory drug, a vasopressor, and/or an antibody or antibody fragment. Suitable small peptides include macrocyclic peptides, θ defensins, and/or θ defensin analogs. Such a small peptide can be resistant to a microbial protease.

Small peptides suitable for such compositions include cyclic peptides can have a structure as shown in FIG. 2A, where AA3 and AA12 are cysteines joined by a disulfide bond, AA5 and AA10 are cysteines joined by a disulfide bond, AA4 is a first hydrophobic amino acid, AA11 is a second hydrophobic acid, AA6 is arginine, AA7 is arginine, AA8 is arginine, and wherein the cyclic peptide has four arginine residues that provide a positively charged content of about 28% at physiological pH. Alternatively, suitable small peptides can include cyclic peptides that have a structure as shown in FIG. 2A, where AA3 and AA12 are cysteines joined by a disulfide bond, AA5 and AA10 are cysteines joined by a disulfide bond, AA4 is serine or a first hydrophobic amino acid, AA11 is serine or a second hydrophobic acid, AA6 is arginine, AA7 is arginine, AA8 is arginine, and wherein the cyclic peptide comprises five arginine residues that provide a positively charged content of at least about 36% at physiological pH.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1: FIG. 1 shows a schematic depiction of exemplary cyclic peptides referred to throughout. RTD-1 (SEQ ID NO: 1) is a naturally occurring octadecapeptide θ-defensin. Remaining peptides are θ-defensins analogs.

FIGS. 2A and 2B: FIG. 2A shows a schematic of a cyclic defensin analog, showing numeric designations for amino acids by position along the cyclic chain. FIG. 2B provides an example of the application of these designations to amino acids of the Peptide 1 (SEQ ID NO: 2) peptide.

FIG. 3: Shows the results of efficacy studies of macrocyclic peptides in a murine carbapenem resistant Klebsiella pneumoniae sepsis model.

FIG. 4: Shows the results of potency studies of Peptide 1 (SEQ ID NO: 2) in a murine carbapenem resistant Klebsiella pneumoniae sepsis model.

FIGS. 5A to 5D: FIGS. 5A to 5D show the effects of Peptide 2 on survival in CRE bacteremic sepsis. In FIGS. 5A to 5C mice were challenged i.p. with Kp-1705 (3-5×10⁸ CFU) at t=0. FIG. 5A shows results when at one hour post infection (p.i.) mice were treated i.p. with Peptide 2 at 0.5, 1.25, 5 mg/kg, or saline vehicle and monitored for up to 28 days. FIG. 5B shows results when mice were treated with a single i.p. injection of 1.25 mg/kg Peptide 2 before (−1 or −3 h) or after (+1 or +3 h) infection; controls received saline. P values are compared to saline control. FIG. 5C shows results when mice were treated 1 hour post infection with a single i.p. dose of 1.25 mg/kg Peptide 2, meropenem, imipenem, colistin, or polymyxin B or 5 mg/kg RTD-1. FIG. 5D shows results when mice were challenged i.p. with CRE E. coli BAA-2340 (3-6×10⁷ CFU). One hour after infection, mice received a single i.p. injection of 5 or 1.25 mg/kg Peptide 2 or saline. P values were determined by Fisher's exact test.

FIGS. 6A to 6C: FIGS. 6A to 6B show rapid dissemination of Kp-1705 following intraperitoneal infection. Male and female BALB/c mice were challenged i.p. with Kp-1705 (3-5×10⁸ CFU). FIG. 6A shows results when at one hour p.i. animals were euthanized and bacterial burden determined for blood, spleen, kidney, liver, lungs/heart, and peritoneal lavage (n=8). FIG. 6B shows results for Bacterial burden in blood at 1, 4, and 24 hours p.i. from similar studies. FIG. 6C shows results from spleen homogenate at 1, 4, and 24 hours p.i. in similar studies (c) were determined Data are means±95% CI.

FIGS. 7A to 7C: FIGS. 7A to 7C show Peptide 2 pharmacokinetics and peritoneal fluid antimicrobial activities. In FIGS. 7A and 7B naïve BALB/c mice (2M/2F per time point) received 1.25 mg/kg Peptide 2 by a single i.p. injection. FIG. 7A shows results from quantitation of the peptide in blood plasma using LC-MS/MS. FIG. 7B shows results from quantitation of the peptide in peritoneal fluid using LC-MS/MS. FIG. 7C shows results from studies in which peritoneal fluid was collected from uninfected mice (2M/2F) injected i.p. with saline or 1.25 mg/kg Peptide 2. Fluids were inoculated in vitro with 2×10⁷ CFU/ml of Kp-1705, incubated for 1 hour at 37° C., and viability determined by counting CFU on TSA plates.

FIGS. 8A to 8E: FIGS. 8A to 8E show that Peptide 2 promotes bacterial clearance, neutrophil recruitment and phagocytosis in Kp-1705 bacteremia. In FIGS. 8A to 8D, BALB/c mice were challenged i.p. with Kp-1705 (3-5×10⁸ CFU) and treated with 1.25 mg/kg Peptide 2 (M) or saline control (C) at 1 hour post infection. FIG. 8A shows results of studies in which mice were euthanized 4 or 24 hours infection and bacterial burden determined. Data are geometric means±95% CI with P values calculated using Student's t-test. FIG. 8B shows results of similar studies in which, at 2, 4, and 24 hours infection, peritoneal lavage fluid was collected and cytospin preparations stained with H & E. FIG. 8C shows results of studies in which peritoneal neutrophils were collected and counted 24 hours infection from sham (S) and Kp-1705 (Kp) infected mice treated 1 hours infection with saline (S) or 1.25 mg/kg Peptide 2 (M). FIG. 8D shows results of studies in which blood neutrophils from infected animals treated 1 hour infection were quantified at t=0, 2, 4 and 24 h, data shown as means±95% CI (n=5 for sham n=9-12 for treatment groups), P values determined by Student's t-test with Welch's correction. FIG. 8E shows results of studies in which naïve BALB/c mice (2M/2F per time point) received 1.25 mg/kg Peptide 2 by a single i.p. injection, and blood neutrophil numbers were determined by CBC analysis. The graph depicts means±SD, P values determined by Student's t-test comparing each time point to t=0.

FIGS. 9A to 9F: FIGS. 9A to 9D show that Peptide 2 promotes phagocytosis of Kp-1705. FIGS. 9A and 9B show results of studies characterizing phagocytosis by RAW 264.7 macrophages incubated with Peptide 2 in the presence of Kp-1705 (50:1 MOI). FIG. 9A shows results of studies in which Cytospins were stained with H&E and phagocytic index (bacteria per cell) was determined manually. Scatter plots depict means±95% CI, P values determined by ANOVA with Uncorrected Fisher's LSD post-test, *P≤0.05, **P<0.01, ***P<0.0001. FIG. 9B provides images comparing effects of 0 and 0.313 μg/ml Peptide 2 on phagocytic activity. FIG. 9C shows results of studies in which mice were challenged i.p. with Kp BAA-1705 cells preincubated for 1 hour prior to infection with saline (●, n=26) or 1.25 μg/ml Peptide 2 (▪, n=26). A control cohort received saline preincubated bacteria followed immediately by i.p. treatment with 500 μl of Peptide 2 at 1.25 μg/ml (▴, n=12). Survival of each cohort is shown. Experiment was repeated 4 times, P values determined by Fisher's exact test. FIG. 9D shows results of studies in which PLF was collected from 5 mice challenged with saline (C) or Peptide 2-pretreated Kp-1705 (as in FIG. 9C) that were euthanized 4 hours post infection, and bacteria in each sample were quantified by plating (means±SD; P values by Student's t-test). FIG. 9E shows results of H & E stained cytospin preparations from saline-pretreated samples. FIG. 9F shows results of H & E stained cytospin preparations from MTD1280-pretreated samples.

FIGS. 10A and 10B: FIGS. 10A and 10B show that sub-MIC concentrations of Peptide 2 do not affect bacterial viability or replication fitness of Kp-1705. Kp-1705 (1×10⁹ CFU/ml) was incubated for 1 hours with 1.25 μg/ml Peptide 2 or saline. FIG. 10A shows results of studies in which bacterial viability was quantified as CFU on TSA plates. P value determined by Student's t-test. FIG. 10B shows results of studies in which Kp-1705 replication fitness was determined by incubating a 40-fold dilution of the incubation mixtures from FIG. 10A in TSB and measuring bacterial growth (black-saline; red-Peptide 2) at A620. The green growth curve is of bacteria incubated with TSB in the presence of 1.25 μg/ml Peptide 2 and “TSB only” is absorbance of sterile medium. Samples were analyzed in triplicate with standard deviation shown.

FIG. 11: FIG. 11 shows Peptide 2 modulation of cytokine responses in Kp-1705 sepsis. Mice were challenged i.p. with Kp-1705 and treated 1 hour later with Peptide 2 (1.25 mg/kg i.p.; M) or saline (C). The sham (S) cohort received bacteria-free suspension buffer at t=0 followed by saline 1 hour later. Mice were euthanized 4 or 24 hours p.i. and plasma samples and cytokines quantified. P values were calculated using ANOVA with Uncorrected Fisher's LSD.

FIG. 12: FIG. 12 shows results of studies of transient stimulation of cytokines by Peptide 2. Sham infected mice were injected i.p. with PBS and treated i.p. 1 hour later with 1.25 mg/kg Peptide 2 (M) or saline (S). Four or 24 hours post sham infection, animals were euthanized, and plasma samples quantified by multiplex cytokine analysis. P values calculated using ANOVA with Uncorrected Fisher's LSD comparing saline and Peptide 2 treated mice.

FIGS. 13A and 13B: FIGS. 13A and 13B show results of studies of in vitro Peptide 2 modulation of cytokine responses. FIG. 13A shows results of studies in which LPS-stimulated RAW 264.7 cells were incubated with Peptide 2 (●) or RTD-1 (▴) for 2 h and supernatant TNF quantified by ELISA. FIG. 13B shows results of studies in which human whole blood was incubated for 4 h with 1000 CFU/ml of Kp-1705 with Peptide 2, and supernatant TNF (●) and IL-6 (▪) were quantified by ELISA.

### DETAILED DESCRIPTION

The inventive subject matter provides synthetic small peptides (i.e., a peptide having a molecular weight of less than about 50 kD, 40 kD, 30 kD, 25 kD, or about 20 kD) that are effective in the treatment of multidrug resistant bacterial infection. Such peptides act by modulating two, three, or more host defense mechanisms.

Within the context of this application, a “subantimicrobial” concentration in regard to a should be understood to be a concentration at which the compound so described has no antimicrobial effect when applied to the a representative microbial pathogen in vitro (e.g. in a liquid culture medium), e.g. in the absence of host immune effectors For example, a subantimicrobial concentration of a compound in regard to Klebsiella pneumoniae would be a concentration that is less than that which demonstrates an antimicrobial effect against the organism in an in vitro setting (e.g. in the absence of host immune effectors). Such subantimicrobial concentrations can be determined experimentally (for example, by culture from a patient sample) or from historical data.

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments are approximations, the numerical values set forth in the specific examples are reported as precisely as practicable. The numerical values presented in some embodiments may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

One should appreciate that the disclosed peptides provide many advantageous technical effects, including providing treatment of infection with multi-drug resistant bacteria when administered in low, sub antimicrobial amounts.

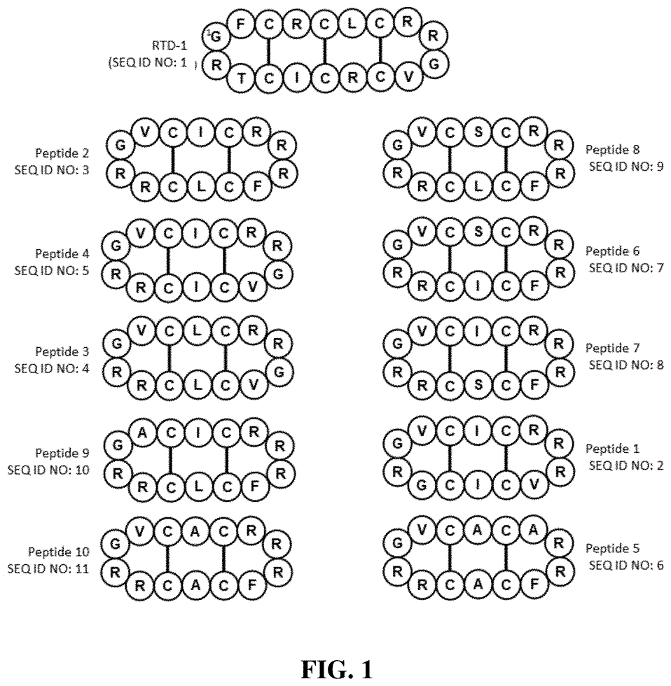

Inventors have described synthetic cyclic tetradecapeptide analogs of the theta defensin RTD-1 (SEQ ID NO: 1) that showed some of the activities of the parent peptide, despite their smaller size and reduced number of disulfide bonds. The structures of natural theta defensin RTD-1 (SEQ ID NO: 1) and some exemplary synthetic cyclic tetradecapeptide analogs are shown in FIG. 1. As shown, RTD-1 (which is expressed naturally in rhesus monkeys) is a cyclic octadecapeptide that includes 3 pairs of cysteines coupled by disulfide bonds that transit the circular primary structure of the peptide. A number of examples of synthetic (i.e., non-naturally occurring) analogs of RTD-1 are shown. Each of the exemplary synthetic analogs is a tetradecapeptide that includes 2 pairs of cysteines coupled by disulfide bonds. These disulfide bonds transit the circular primary structure of the synthetic peptides to form a “box” substructure that incorporates additional amino acids. It should be appreciated that these exemplary analogs show varying degrees of sequence identity with RTD-1, and in some instances show conservative amino acid substitutions near and between the “box” defined by cysteines of the synthetic peptide analogs.

Inventors have developed a novel therapeutic strategy employing a small peptide, for example in the form of a macrocyclic peptide, that is highly effective in a mouse model of CRE bacteremic sepsis. Peptide 2 is an example of such a peptide, and acts by promoting a plurality of host-mediated mechanisms, including (but not limited to) clearance of the pathogen by stimulating phagocytosis, while also homeostatically modulating dysregulated systemic inflammation. It should be appreciated that the effects of such host mechanisms are not limited to bacterial infection (as provided in examples below), but are also effective against fungal, viral, and protozoan infection. Accordingly, Inventors contemplate effects against a range of microbes that includes bacteria, fungi, viruses, and/or protozoans.

Peptide 2 is a 14-amino acid macrocyclic peptide containing two disulfide bonds (FIG. 1) that possesses features of naturally occurring macrocyclic θ-defensins expressed uniquely in myeloid and epithelial cells of Old World monkeys (OWM, e.g., rhesus macaques, baboons, vervets, cynomolgus monkeys)^{8,9} but not in other primates or non-primates. All known natural θ-defensins are 18-amino acid cyclic peptides containing three disulfides which confer remarkable stability in vitro and in vivo¹¹⁻¹⁴. Studies show that θ-defensins possess potent antimicrobial activities in vitro and unique anti-infective and immunoregulatory properties in vivo. The prototype rhesus macaque θ-defensin RTD-1 (FIG. 1) is effective in preclinical models of severe sepsis and associated shock. It has been hypothesized that θ-defensins contribute to the unique host defense and immunomodulatory responses of ole world monkeys, primates that, compared to humans and other hominins, are intrinsically resistant to bacterial infections, endotoxemia, and sepsis. In sepsis models, θ-defensins facilitate host-mediated clearance of bacterial and fungal pathogens while moderating cytokine-driven immunopathology.

On investigation, Peptide 2 emerged as a promising lead for further preclinical evaluation as a therapeutic for multi-drug resistant (MDR) infections. Peptide 2 was found to be efficacious in treatment of CRE sepsis in BALB/c mice and acts by stimulating host-mediated clearance of CRE pathogens through enhancement of a plurality of host defense responses, including phagocytosis and immune cell recruitment, while concomitantly modulating pathogenic cytokine responses (as shown below).

Peptide 2 is a bioinspired macrocyclic peptide that is structurally related to naturally occurring θ-defensins. Among a series of minimized θ-defensins, Peptide 2 was identified as a promising candidate based on its low toxicity in vitro and in vivo, stability in biological matrices, and suppression of TNF release by LPS-stimulated THP-1 macrophages. As shown herein, Peptide 2 is highly effective in promoting long term survival, bacterial clearance, and moderation of dysregulated systemic inflammation in CRE septicemia.

Both therapeutic and prophylactic single dose administration of Peptide 2 significantly enhanced long term survival following challenge with Kp-1705, an organism that is resistant to carbapenems both in vitro and in vivo. Moreover, Peptide 2 was as effective in vivo as colistin and polymyxin B, drugs of last resort that have limited utility as human therapeutics owing to their toxicities. RTD-1 was also effective in Kp-1705 sepsis, but at approximately 10-fold higher concentrations than the effective dose of Peptide 2. In addition, Peptide 2 promoted long term survival in mice challenged with a CRE strain of E. coli (BAA-2340) demonstrating efficacy against the two most common species of CRE pathogens.

Highly efficacious single dose treatment of both CRE pathogens was obtained with administration of Peptide 2 at 1.25 mg/kg, a dose level that resulted in plasma Cmax of 0.25 μg/ml which is >400 times lower than the MIC of Peptide 2 against both organisms in the presence of serum (>100 μg/ml). Moreover, Peptide 2-containing peritoneal fluid had no bactericidal activity against Kp-1705. This demonstrates that the antimicrobial effect of Peptide 2 is not direct, but rather is mediated by stimulation of host clearance mechanisms, similar to the therapeutic effect of RTD-1 in systemic candidiasis 15.

In the bacteremia model employed, Kp-1705 was widely disseminated in blood and solid organs at the time of Peptide 2 treatment. This, and the lack of Peptide 2 activity in peritoneal fluid, enabled infection and delayed peptide administration by the same route. Peptide 2 treatment induced marked reductions in bacterial burden in blood, peritoneal lavage, and spleen within 4 hours of treatment, and this was accompanied by a marked peptide-induced neutrophilic infiltrate in the peritoneal cavity. Microscopic analysis of PLF from Peptide 2 treated infected mice revealed markedly increased phagocytosis of bacteria compared to control. Blood neutrophil levels were also transiently elevated in infected mice treated with Peptide 2 as well as in mock-infected controls, suggesting that the peptide stimulates a systemic neutrophilic response. Of note, i.p. administration of Peptide 2 induced transient rises in CXCL1, IL-5, and IL-6 in sham treated mice. CXCL1 is a potent neutrophil chemokine which in concert with its receptor CXCR2, plays a key role as one of two cytokine/receptor axes controlling the migration of neutrophils from the bone marrow 29. Thus, CXCL1 may mediate Peptide 2-induced transient neutrophilia.

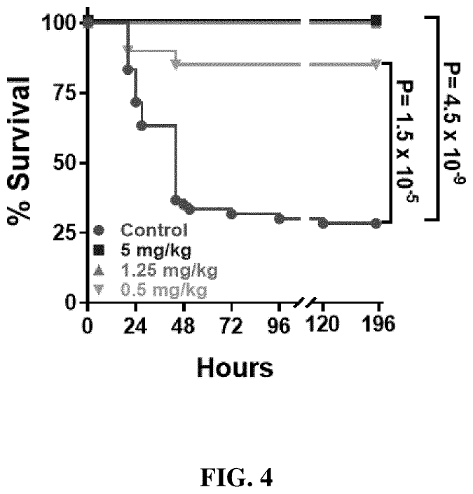

Peptide 2-mediated phagocytosis, revealed by cytologic examination of PLF in infected mice, was recapitulated in vitro by co-incubation of Kp-1705 with RAW 264.7 macrophages in the presence of nanomolar concentrations of the peptide, resulting in up to 12-fold increases in phagocytic index. Consistent with this, preincubation of Kp-1705 with a sub-MIC concentration of Peptide 2 promoted phagocytosis in vivo and markedly enhanced mouse survival (FIG. 4c), evidence that Peptide 2 has opsonin-like properties. Mice were also protected from Kp-1705 septicemia by a single dose of Peptide 2 administered 1 or 3 hours prior to infection, indicating that systemic levels of the peptide are also effective prophylactically. The degree to which this effect is mediated by induction of phagocytosis in extraperitoneal tissues remains to be determined.

Pathogenesis of septicemia results from an imbalance of the host damage-response framework. Challenge with K. pneumoniae initiates host innate immune responses which progress through systemic inflammatory response syndrome, evolving to severe sepsis, and finally in lethal septic shock. This detrimental host response is largely driven by dysregulated cytokine signaling which results in uncontrolled inflammation, host-mediated tissue destruction, disseminated intravascular coagulation, and multiple organ failure. Modulation of cytokine responses in Peptide 2 treated mice was associated with suppression of otherwise immunopathologic cytokine release without inducing an immunocompromised state. Moreover, Peptide 2 treatment significantly enhanced the recruitment of neutrophils to the peritoneal cavity, an innate immune response which is often accompanied by inflammatory tissue damage. However, Peptide 2-induced neutrophil recruitment was accompanied by increased phagocytosis, bacterial clearance, and homeostatic resolution of plasma cytokines.

Although mouse models of bacteremic sepsis are distinct from human sepsis syndromes, they are a common starting point for identification of infectious disease drug candidates. To address the potential of Peptide 2 as a human therapeutic, the immunomodulatory activity of the peptide on TNF and IL-6 expression by bacteria-stimulated human blood leukocytes was analyzed. The peptide suppressed release of both cytokines in a concentration dependent manner. Other experiments showed that Peptide 2 is remarkably stable in plasma and when incubated for 24 hours with live CRE isolates, suggesting that this macrocyclic peptide is an attractive candidate for further preclinical development. Additional studies showed that i.p. administration of Peptide 2 is well tolerated in BALB/c mice at doses more than 100-fold higher than the 0.5 mg/kg efficacious dose (data not shown).

It has been found that θ-defensin-inspired macrocyclic small peptides such as Peptide 2 are remarkably effective for treatment of Gram negative bacterial infections. Like RTD-1, the mechanism of action of Peptide 2 is host directed, and therefore, less likely to select for drug resistance than conventional antibiotics. Moreover, the stability and pharmacologic properties of Peptide 2 suggests that it represents a promising new class of immunomodulating anti-infectives much needed to address the worldwide crisis resulting from antimicrobial resistance.

A series of θ-defensin analogs have been prepared and screened. These analogs were extrapolated from the θ-defensin analog designated Peptide 2 (see FIG. 1, SEQ ID NO: 3), a synthetic peptide that provides substantially improved effects (relative to RTD-1) in long term survival of mice in a model of sepsis, and that provides these effects at surprisingly low concentrations. It should be appreciated that long term survival of sepsis requires both management of the infecting organism and of the shock induced by the host response to the infection, either of which can lead to death.

Surprisingly, θ-defensin analogs were identified that can also provide a biphasic response in modulating the immune system. The initial effect is opsonic, recruiting effector cells to the sepsis site. This serves to combat infection, and surprisingly was found to occur at concentrations of the θ-defensin analog that demonstrated neither a bactericidal nor a bacteriostatic effect (i.e., subantimicrobial concentrations). Following this initial opsonic effect these synthetic θ-defensin analogs exhibit a longer term immunomodulatory effect (for example, reducing TNF, IL-6 and other inflammatory cytokines) that contributes to long term survival in preventing septic shock.

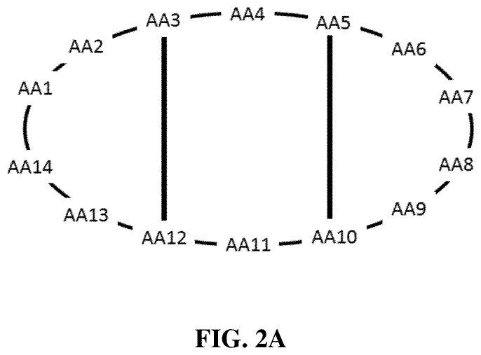

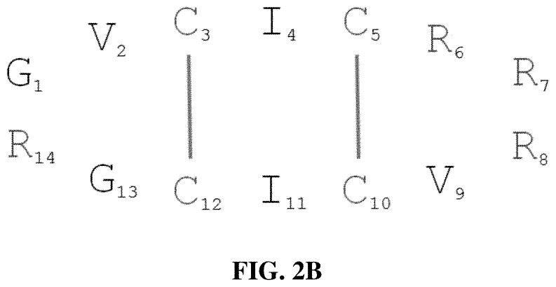

As noted above, examples of a naturally occurring θ-defensin and exemplary θ-defensin analogs are shown in FIG. 1. It should be appreciated that these are cyclic peptides that lack conventional amino- and carboxyl-termini; as such amino acid sequence information as provided in accompanying amino acid sequence listings should not be construed as based on a discrete N-terminus or C-terminus. The primary structure of the naturally occurring θ-defensin RTD-1 (SEQ ID NO: 1) is shown at the top of FIG. 1. The remaining peptides are exemplary non-natural analogs of θ-defensins. In the 14-amino acid analog series, it should be appreciated their three dimensional structures include a first β-turn formed by amino acids 6 to 9 and a second β-turn formed by amino acids 13, 14, 1, and 2 as designated using a numbering system adapted for use with cyclic θ-defensins and their analogs and as shown in FIGS. 2A and 2B.

Although these cyclic peptides do not have free amino- or carboxyl-termini, amino acid positions within the cyclic structure can be designated based on their positions relative to certain structural features (such as disulfide bonds and/or a distinctive ‘triplet’ of arginines). Such a set of designations as utilized for this purpose within this application is illustrated in FIG. 2A, where amino acids are designated 1 to 14 (AA1, AA2, etc.) in a cyclic teradecapeptide structure having a circular and continuous chain of peptide bonds through the primary amines of the individual amino acids (i.e. not through side chain groups), and in which two intra-peptide covalent bonds occur between side chains of cysteine amino acids designated AA3 and AA12 and between side chains of cysteine amino acids designated amino acids AA5 and AA10. FIG. 2B depicts application of amino acid positions as within the context of this application, as applied to an exemplary synthetic cyclic tetradecapeptide (Peptide 1 peptide, SEQ ID NO: 2). Amino acid identity is designated using single-letter amino acid code.

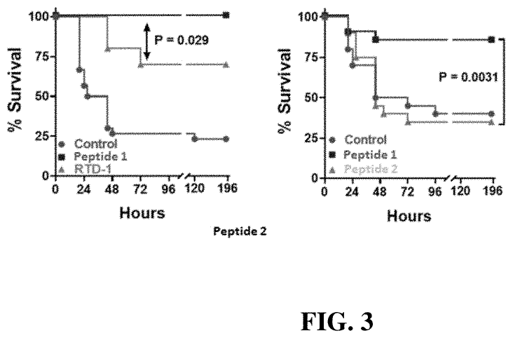

FIG. 3 shows the results of studies of survival of septic shock following application of RTD-1 (SEQ ID NO: 1) and exemplary novel synthetic tetradecapeptides in a murine model of sepsis utilizing an antibiotic-resistant bacteria that results in 75% mortality (i.e., 25% survival) if untreated. BALB/c mice were infected interperitoneally with 3-5×10⁸ CFU of a carbapenem resistant strain of Klebsiella pneumoniae (KPC+-Kp BAA-1705 (ATCC)) and treated with peptide one hour post infection. The left panel of FIG. 3 shows exemplary results of comparative studies between the synthetic peptide Peptide 1 (SEQ ID NO: 2) and naturally occurring RTD-1 administered at 5 mg/kg. The right panel of FIG. 3 shows exemplary results of comparative studies between the synthetic peptides Peptide 1 and Peptide 2 (SEQ ID NO: 3) administered at 0.5 mg/kg. P-values were determined by Fisher's exact test. The therapeutic peptides were provided intraperitoneally 1 hour after induction of sepsis.

As shown in the left panel of FIG. 3, at 5 mg/kg RTD-1 (SEQ ID NO: 1) provides only partial protection (70% survival vs 25% for sham controls), whereas Peptide 1 (SEQ ID NO: 2) provides complete protection from sepsis and septic shock. As shown in the right panel of FIG. 3, at 0.5 mg/kg the effects of Peptide 2 (SEQ ID NO: 3) on survival are essentially identical to that of the sham control, whereas Peptide 1 provides almost 90% survival. This difference is highly significant (P=0.0031).

FIG. 4 shows typical results for survival studies in such a murine survival study for various concentrations of Peptide 1 (SEQ ID NO: 2), utilizing the same murine sepsis model as used in studies shown in FIG. 3. BALB/c mice were infected i.p. with KPC+-Kp BAA-1705 (ATCC) and treated with a single dose of Peptide 1, at the levels indicated, 1 hour post infection. The significance (P-values determined by Fisher's exact test) of the therapeutic effect for each dose is shown. As shown, 1.25 mg/kg of this novel θ-defensin analog is as effective as 5 mg/kg, and a dose of Peptide 1 as low as 0.5 mg/kg is also highly effective. It should be appreciated that 0.5 mg/kg is the lowest dose tested, and that Inventors believe that Peptide 1 is effective at still lower doses, for example down to 0.25 mg/kg, 100 μg/kg, 50 μg/kg, 25 μg/kg, or 10 μg/kg.

As noted above, Peptide 1 (SEQ ID NO: 2) was identified in screening studies of a range of cyclic peptide analogs of the θ-defensin RTD-1 (SEQ ID NO: 1). RTD-1 is a cationic, arginine-rich cyclic peptide that includes 18 amino acids and 3 disulfide bonds between pairs of cysteines (FIG. 1). Other active θ-defensin analogs are also shown in FIG. 1.

A number of novel θ-defensin analogs have been identified that show superior performance relative to Peptide 2 (SEQ ID NO: 3), despite having similar covalent structures (e.g., length, cyclic configuration, two pairs of disulfide bonds, and cationic character). Features evaluated included survival efficacy in antibiotic resistant K. pneumoniae sepsis, biocompatibility (lack of toxicity), in vitro suppression of TNF-α (TNF) release, and inhibition of TACE. Amino acid sequences of exemplary cyclic peptides are shown in Table 1. It should be appreciated that amino acids identities are indicated using the numerical designation for corresponding positions within the cyclic structures as established in FIG. 2. Properties and activities associated with these peptides are shown in Table 2

TABLE 1

| Analog | | | | 1^{st} β turn | | | | | | | 2^{nd} β turn | | | | SEQ |
|---|---|---|---|---|---|---|---|---|---|---|---|---|---|---|---|
| name | 3 | 4 | 5 | 6 | 7 | 8 | 9 | 10 | 11 | 12 | 13 | 14 | 1 | 2 | ID NO: |
| Peptide 1 | C | I | C | R | R | R | V | C | I | C | G | R | G | V | 2 |
| Peptide 2 | C | I | C | R | R | R | F | C | L | C | R | R | G | V | 3 |
| Peptide 3 | C | L | C | R | R | G | V | C | L | C | R | R | G | V | 4 |
| Peptide 4 | C | I | C | R | R | G | V | C | I | C | R | R | G | V | 5 |
| Peptide 5 | C | A | C | A | R | R | F | C | A | C | R | R | G | V | 6 |
| Peptide 6 | C | S | C | R | R | R | F | C | I | C | R | R | G | V | 7 |
| Peptide 7 | C | I | C | R | R | R | F | C | S | C | R | R | G | V | 8 |
| Peptide 8 | C | S | C | R | R | R | F | C | L | C | R | R | G | V | 9 |
| Peptide 9 | C | I | C | R | R | R | F | C | L | C | R | R | G | A | 10 |
| Peptide 10 | C | A | C | R | R | R | F | C | A | C | R | R | G | V | 11 |

Amino acid positions are designated according to the convention shown in FIG. 2A.

TABLE 2
Properties and activities of cyclic peptide analogs
of the θ-defensin RTD-1 as listed in Table 1.

| | | | Kp sepsis | TACE | TNF % |
|---|---|---|---|---|---|
| Analog | Positive | Molecular | % survival | IC₅₀ | suppression |
| name | charge | Weight | at 5 mg/kg | μg/mL | at 5 mg/kg |
| Peptide 1 | 4 | 1572 | 100 | 0.858 | 95.6 |
| Peptide 2 | 5 | 1720 | 80-100 | 2.202 | 98.7 |
| Peptide 3 | 4 | 1572 | 53.3 | 1.150 | 78.8 |
| Peptide 4 | 4 | 1572 | 40 | 0.546 | 83.0 |
| Peptide 5 | 4 | 1550 | toxic | 1.828 | not tested |
| Peptide 6 | 5 | 1693 | 30 | 3.192 | 37.5 |
| Peptide 7 | 5 | 1693 | 20 | 1.869 | 14.9 |
| Peptide 8 | 5 | 1693 | 0 | 2.613 | 2.0 |
| Peptide 9 | 5 | 1691 | toxic | 0.515 | not tested |
| Peptide 10 | 5 | 1635 | toxic | 2.737 | not tested |

Inventors further believe that members of such a class of θ-defensin-inspired macrocyclic peptides having the following characteristics can provide effects that are similar or identical to those demonstrated by Peptide 2:
Two disulfide bonds, between Cys3 and Cys12 and between Cys5 and Cys10, respectively.
A hydrophobic amino acid positioned between Cys3 and Cys5 and a hydrophobic amino acid positioned between Cys10 and Cys12 in the primary structure of the θ-defensin analog (i.e. at positions 4 and 11), preferably leucine or isoleucine. In combination with the disulfide bonds noted above this defines a feature referred to as the “C-X-C box” within the circular primary structure of the peptide, where “C” is a cysteine and “X” is preferably either leucine or isoleucine.

In some embodiments, a C-X-C box that includes arginine.
A total of four arginine residues that provide the peptide with a charge of +4 at physiological pH.
A triplet of adjacent arginines at positions 6, 7, and 8, (i.e. within the first β-turn).

In some embodiments such θ-defensin-inspired macrocyclic peptides can also include one or more of the following features:
A glycine at position 1 and a glycine at position 13.
Hydrophobic amino acids at position 2 and position 9, preferably valine or phenylalanine.
An arginine within the second β-turn (e.g. at position 14).

Inventors further believe that toxicity of such of θ-defensin-inspired macrocyclic peptides can be reduced or eliminated by not including one or more of:
An alanine at position 4.
An alanine at position 11.

Alternatively or in addition, suitable peptides can have the following characteristics:
Two disulfide bonds, between Cys3 and Cys12 and between Cys5 and Cys10, respectively.
A hydrophobic amino acid or serine positioned between Cys3 and Cys5 and a hydrophobic amino acid or serine positioned between Cys10 and Cys12 in the primary structure of the θ-defensin analog (i.e. at positions 4 and 11), where the hydrophobic amino acid is preferably leucine or isoleucine. In combination with the disulfide bonds noted above this defines a feature referred to as the “C-X-C box” within the circular primary structure of the peptide, where “C” is a cysteine and “X” is leucine, isoleucine, or serine.
A total of five arginine residues that provide the peptide with a charge of +5 at physiological pH.
A triplet of adjacent arginines at positions 6, 7, and 8, (i.e., within the first β-turn).
   In some embodiments suitable peptides can also include one or more of the following features:
A glycine at position 1.
Hydrophobic amino acids at position 2 and position 9, preferably valine or leucine.
An arginine pair within the second β-turn (e.g., at positions 13 and 14).

Toxicity of candidate peptides suggests that active θ-defensin analogs should not include one or more of:
An alanine at position 4.
An alanine at position 11.

Accordingly, Inventors believe a synthetic cyclic tetradecapeptide θ-defensin analog that include a “C-X-C box” structure as described above, a triplet of adjacent arginine residues at positions 6, 7, and 8, a hydrophobic amino acid (e.g., valine or phenylalanine) at position 9, and having a net positive charge of +4 or +5 (about 28% or about 36% of total amino acid content) due to arginine content can provide activities in regard to host responses mechanisms that correspond to those of Peptide 2.

Accordingly, Inventors believe a θ-defensin analog that include a “C-X-C box” structure as described above, a triplet of adjacent arginine residues at positions 6, 7, and 8, a hydrophobic amino acid (e.g., valine or phenylalanine) at position 9, and having a net positive charge of +4 or +5 (about 28% to about 36% of total amino acid content) due to arginine content can be effective in treating multi-drug resistant bacterial infections.

Such small peptides can be effective when administered in amounts that are effective to provide a serum or plasma concentration of from about 0.01 μg/mL to 100 μg/mL (or any range of values therein) following administration. Alternatively, such small peptides can be effective when administered at about 10 μg/kg to about 10 mg/kg (or any range of values therein) following administration. Such administration can be by any suitable method. Suitable methods include, but are not limited to, injection (e.g., intraperitoneal injection, intradermal injection, intravascular injection, intramuscular injection, infusion, etc.), topical application (e.g. to a mucous membrane, skin surface, etc.), inhalation, and/or inhalation.

Such small peptides can be applied on any suitable schedule. For example, such small peptides can be provided as single dose or can be provided as repeated doses. Repeated doses can be provided at any suitable frequency. For example, repeated doses can be provided hourly, every two hours, 8 times a day. 6 times a day, 4 times a day, 3 times a day, twice a day, daily, every two days, every 3 days, weekly, every 10 days, every two weeks, every three weeks, monthly, every two months, every three months, quarterly, every 6 months, annually, or any interval between such boundaries. The frequency, administrative mode, and dosage of such small peptides can be adjusted during treatment.

It should be appreciated that at the dosages applied in such studies the θ-defensin and/or its analog does not produce a drug Cmax sufficient to have an appreciable direct antimicrobial (e.g. bactericidal, bacteriostatic) effect. Without wishing to be bound by theory, Inventors believe that the antimicrobial effects of Peptide 1 (SEQ ID NO: 2) characteristic of the first phase of the biphasic response are the result of a systemic immune activating mechanisms that result in recruitment and stimulation of cells from the host immune system, which in turn phagocytose, kill, and clear the pathogen.

### Examples

In vitro antibacterial activity of Peptide 2. Minimum inhibitory concentrations (MICs) of Peptide 2 and RTD-1 against a panel of CRE bacteria were determined and compared with carbapenem antibiotics (meropenem and imipenem), colistin, and polymyxin B. Test organisms included K. pneumoniae and E. coli strains expressing carbapenemases (KPC-2, NDM-1), extended-spectrum β-lactamase (ESBL) resistance factors as well as antibiotic sensitive reference strains (see Methods). Under standard CLSI assay conditions, the MIC of Peptide 2 was 3.13-6.25 μg/ml against CRE and non-CRE strains of K. pneumoniae and E. coli. Peptide 2 was superior to RTD-1, and was as or more effective than meropenem and imipenem against each of the strains tested. As expected, colistin and polymyxin B were effective at low concentrations (<0.78 μg/ml) against all seven organisms. Of note, inclusion of 50% mouse serum completely inhibited the activities of Peptide 2 and RTD-1 against KPC+Kp BAA-1705 (Kp-1705) (MIC>100 μg/ml), a finding consistent with a recent study of the antifungal activity of RTD-1¹⁵. In contrast, the antimicrobial activities of the conventional antibiotics tested were unaffected or slightly enhanced by addition of serum (Table 3).

TABLE 3

| | K. pneumoniae | | | | | | | |
|---|---|---|---|---|---|---|---|---|
| | Strain ID | | | | | | | |
| | BAA-1705 | | BAA-2146 | | 700603 | | BAA-1706 | |
| | MDR Factor | | | | | | | |
| | KPC-2 | | NDM-1 | | ESBL | | none | |
| Peptide 2 | 6.25 | >100* | 6.25 | >100* | 6.25 | >100* | 3.13 | >100* |
| RTD-1 | >100 | >100* | 100 | >100* | >100 | >100* | >100 | >100* |
| meropenem | 50 | 50* | 100 | >100* | <0.78 | <0.78* | 1.56 | <0.78* |
| imipenem | >100 | 12.5* | >100 | 25* | <0.78 | <0.78* | 3.13 | <0.78* |
| colistin | <0.78 | <0.78* | <0.78 | <0.78* | <0.78 | <0.78* | <0.78 | <0.78* |
| polymyxin B | <0.78 | <0.78* | <0.78 | <0.78* | <0.78 | <0.78* | <0.78 | <0.78* |

| | E. coli | | | | | |
|---|---|---|---|---|---|---|
| | Strain ID | | | | | |
| | BAA-2340 | | BAA-2471 | | ML35 | |
| | MDR Factor | | | | | |
| | KPC-2 | | NDM-1 | | none | |
| Peptide 2 | 6.25 | >100* | 3.13 | 100* | 3.13 | >100* |
| RTD-1 | 50 | >100* | 25 | >100* | 25 | >100* |
| meropenem | 6.25 | 6.25* | 50 | >100* | <0.78 | <0.78* |
| imipenem | 25 | 6.25* | 50 | 25* | <0.78 | <0.78* |
| colistin | <0.78 | <0.78* | <0.78 | <0.78* | <0.78 | <0.78* |
| polymyxin B | <0.78 | <0.78* | <0.78 | <0.78* | <0.78 | <0.78* |

*MICs in the presence of 50% heat inactivated mouse serum

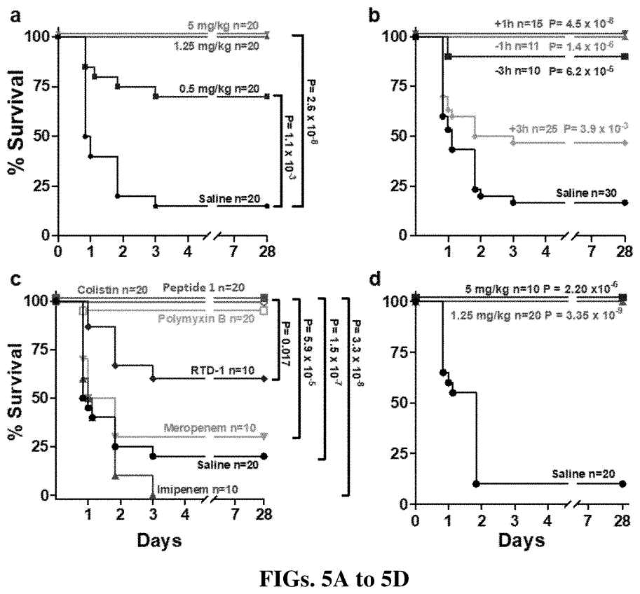

Peptide 2 promotes survival in MDR bacterial septicemia. Adult BALB/c mice were infected intraperitoneally with Kp-1705, a virulent hypermucoid strain of K. pneumoniae, which results in rapid bacterial dissemination to blood, spleen, and other solid organs (liver, lungs, and kidneys; 10⁶-10⁸ CFU/g) within 1 hour of infection. Kp-1705 infected mice showed clinical signs of systemic inflammatory response syndrome (SIRS) 25, including diminished physical activity, hunched posture, piloerection, and altered breathing rates within 3 hours of infection, and ˜75% of vehicle control mice succumbed or were humanely euthanized by 96 hours post infection (p.i.). Peptide (0.5, 1.25, or 5 mg/kg Peptide 2) or saline treatment was initiated 1 hour p.i. A single dose of Peptide 2 at 1.25 and 5 mg/kg dose levels resulted in 100% long term (>28 d) survival (P=2.6×10⁻⁸) (FIG. 5A). Compared to saline-treated controls, the clinical appearance/activity of mice treated with Peptide 2 at 1.25 or 5 mg/kg markedly improved within 24 hours of treatment, and behavior and appearance were normal by 96 hours. Treatment efficacy, measured by survival, was reduced when the dose of Peptide 2 was reduced to 0.5 mg/kg, but survival benefit was still statistically significant compared to saline treatment (P=1.1×10⁻³).

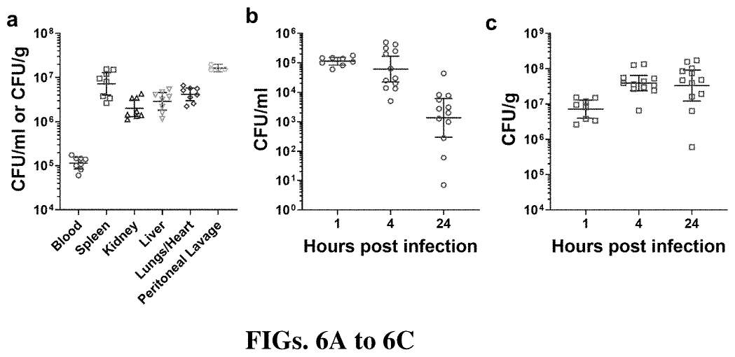

The effect of timing of peptide treatment on Kp-1705 infected mice was analyzed in experiments in which a single 1.25 mg/kg Peptide 2 was administered 1 or 3 hour(s) before or after i.p. infection (FIG. 5B). All infected mice in the −1 hour and +1 hour treatment cohorts survived long term (P<1×10⁻⁶), and nearly equivalent efficacy was obtained when mice were treated 3 hours prior to infection. Even when treatment was initiated 3 hours after infection, survival was enhanced (FIG. 2b; P=3.9×10⁻³). Notably, all mice in the +3 hours cohort showed clinical signs of SIRS (described above) before Peptide 2 treatment, consistent with the high tissue burdens of Kp-1705 in the blood and organs of infected mice (FIGS. 6A to 6C). Moreover, as discussed below, plasma levels of proinflammatory cytokines were markedly elevated within 2-4 hours of Kp-1705 infection in mice, consistent with severe sepsis. Nevertheless, half of the +3 hours cohort were rescued by a single dose of Peptide 2 and were long term survivors.

Comparison of Peptide 2 and conventional antibiotics for efficacy in Kp-1705 septicemia. Equal single doses of Peptide 2, imipenem, meropenem, colistin, and polymyxin B were administered i.p. 1 hour after Kp-1705 challenge (as in FIGS. 5A and 5B). Peptide 2 and colistin treatments resulted in 100% survival, and polymyxin B was 95% effective, but neither carbapenem provided significant survival benefit compared to saline treated controls. Lack of imipenem and meropenem benefit was expected given that Kp-1705 is highly resistant to carbapenems. Surprisingly, Peptide 2 showed superior efficacy at a dose of 1.25 mg/kg compared to 5 mg/kg of natural θ-defensin RTD-1 in vivo (FIG. 5C).

Efficacy of Peptide 2 against MDR E. coli. After Klebsiella spp., E. coli is the second most common cause of CRE infections 1. Given the efficacy of Peptide 2 against K. pneumoniae, Inventors believed that this peptide would be efficacious in E. coli septicemia. Mice were challenged i.p. with KPC+E. coli BAA-2340 and treated 1 hour p.i. with a single injection of peptide at 1.25 or 5 mg/kg. As shown in FIG. 5D, 100% of mice in both Peptide 2 treatment cohorts survived >28 days (P≤2.2×10⁻⁶) compared to 10% survival in saline controls. As observed with Peptide 2 treatment of Kp-1705 infected mice, peptide treated mice infected with E. coli BAA-2340 were healthier than saline controls 24 hours p.i., and appearance and behavior were normal by 96 hours. It is contemplated that this demonstrates effectiveness of Peptide 2 and other members of this class of synthetic peptides (as detailed below) against a broad range of multidrug resistant microbial species.

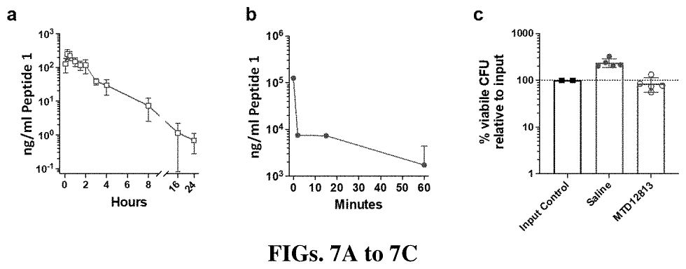

Peptide 2 pharmacokinetics (PK). As noted above, i.p. infection with Kp-1705 results in rapid dissemination into blood and tissues within 60 minutes. Since Peptide 2 treatment initiated 1 or 3 hour(s) post infection is highly effective (FIG. 5B), it is evident that therapeutic efficacy occurs within the setting of widely disseminated infection. To understand the kinetics of peptide absorption and systemic distribution, single dose PK analysis was performed following administration of 1.25 mg/kg of Peptide 2 in naïve male and female BALB/c mice. Peptide 2 was rapidly absorbed from the peritoneum with Tmax of 15 minutes post injection and a peak plasma concentrations (Cmax) of 0.251±0.0798 μg/ml (FIG. 7A). Consistent with these results, rapid peritoneal uptake of Peptide 2 from the peritoneal fluid (PF) was measured, as the 125 μg/ml peptide infusate was reduced to ˜7.5 μg/ml within 2 minutes of ip administration (FIG. 7B). Of note, no bacterial killing was observed in vitro when Kp-1705 was incubated in PF containing 7.5 μg/ml peptide (FIG. 7C), consistent with an indirect mode of action of Peptide 2.

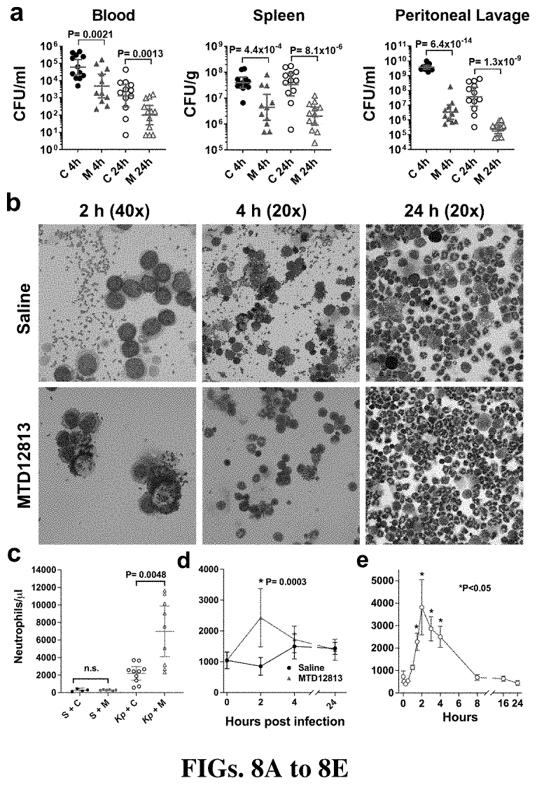

Peptide 2 promotes bacterial clearance, phagocytosis, and neutrophil recruitment in Kp-1705 septicemia. Organs from long term survivors (28 days) of Peptide 2-treated, Kp-1705 infected mice were found to lack culturable bacteria, demonstrating that survival is associated with bacterial clearance. The effect of Peptide 2 on bacterial clearance was evaluated in blood, spleen, and peritoneal lavage fluid (PLF) from mice 4 or 24 hours after infection following a single dose treatment with saline or 1.25 mg/kg Peptide 2 one hour p.i. (FIG. 8A). Peptide 2 treatment significantly reduced bacterial burden in all three tissues at both 4 and 24 hours (P<0.003) (FIG. 8A). Within 3 hours (4 hours p.i.) of Peptide 2 treatment, viable bacteria were reduced by 79.1%, 70.4%, and 99.5% in blood, spleen, and PLF, respectively, compared to saline treated animals. By 24 hours, at which time Peptide 2 treated mice showed marked improvements in clinical appearance and activity, bacterial burdens were further reduced by 93.8% in blood, 94.4% in spleen homogenates, and 99.8% in PLF, demonstrating that Peptide 2 rapidly promotes bacterial clearance.

To further evaluate the effect of peptide treatment on bacterial clearance, Cytospin® preparations of PLF of Kp-1705 infected mice were evaluated 2, 4, or 24 hours p.i. in Peptide 2 and saline treated mice. Compared to saline controls, Peptide 2-treated mice showed a marked increase in peritoneal cell associated or phagocytosed bacteria, and few extracellular bacteria were evident (FIG. 8B). Additionally, peptide treatment promoted a significant increase in peritoneal neutrophils 4 hours p.i. (data not shown) which progressed to a 3-fold increase in PLF neutrophils at 24 hours (P=0.0048; FIG. 8C). The peptide-induced neutrophilic infiltrate was only observed in infected mice, as Peptide 2 treatment of sham-infected mice showed no increase in neutrophil numbers 24 hours after peptide delivery (FIG. 8C). In parallel, a transient peripheral blood neutrophilia occurred in infected mice treated with Peptide 2 (but not saline) which peaked at ca. 2 hours p.i. (FIG. 8D). By 4 hours p.i., blood neutrophil counts in Peptide 2-treated mice were not significantly different from those of mice treated with saline (FIG. 8C). Modest, but not statistically significant, elevations in monocytes at 2 hours p.i. were observed in Peptide 2 treated mice, while lymphocyte, eosinophil, and basophil levels showed no apparent differences among treatment groups. Interestingly, a similar transient neutrophilia was observed in blood collected from naïve BALB/c mice treated with a single i.p. dose of Peptide 2 (FIG. 8E).

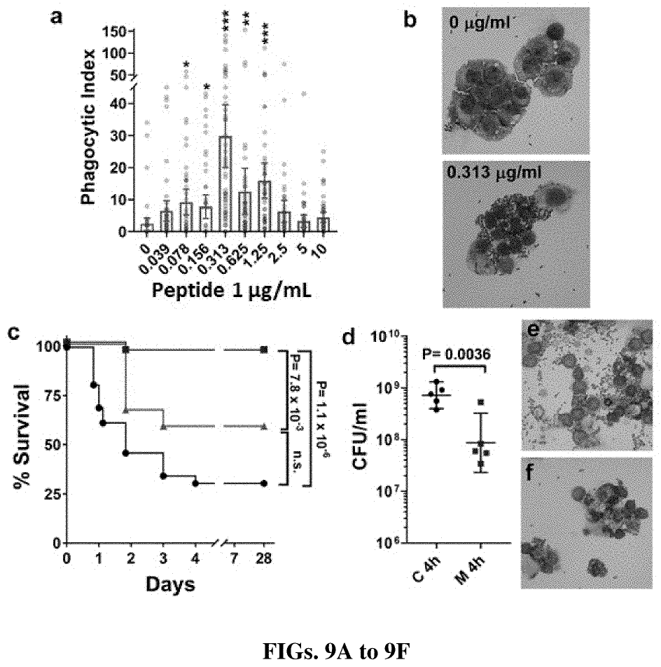

Peptide 2 promotes bacterial phagocytosis. To further analyze the effect of Peptide 2 on phagocytic activity, Inventors analyzed the effect of the peptide on phagocytosis of Kp-1705 by mouse RAW 264.7 macrophages. Co-incubation of live Kp-1705 with RAW 264.7 cells with 0-10 μg/ml Peptide 2 showed a concentration dependent inverted-U induction of phagocytosis (FIG. 9A). Maximal phagocytic activity was observed at 0.313 μg/ml, which resulted in a 12.6-fold increase in mean phagocytic index (P<1×10⁻⁶; FIG. 9A) relative to no peptide controls, and the increase was readily observed microscopically (FIG. 9B). Of note, maximal phagocytic activity was achieved at peptide levels approximating the plasma Cmax values obtained following i.p. administration of 1.25 mg/kg Peptide 2 (FIGS. 7A to 7C).

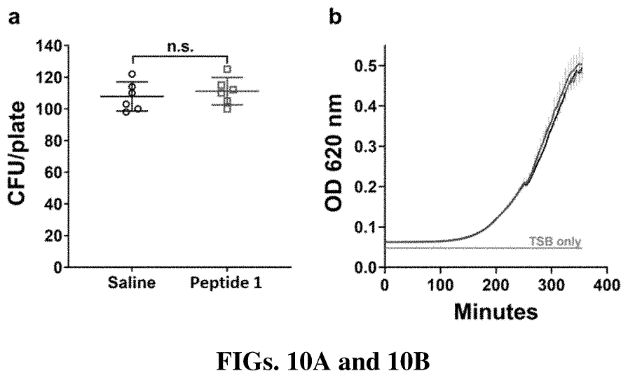

The phagocytosis-inducing activity of Peptide 2 was further analyzed by incubating live Kp-1705 cells with 1.25 μg/ml of Peptide 2 (⅕ the MIC under optimal conditions; Table 3) and challenging mice with Peptide 2 pre-treated bacteria. Although pre-treatment had no effect on bacterial viability or replication fitness of the organism (FIGS. 10A and 10B), mice challenged with the bacteria-peptide mixture were protected (FIG. 9C). Because no statistically significant protection was afforded by i.p. administration of an equal quantity of peptide (0.5 ml of 1.25 μg/ml) immediately following infection, it was inferred that preincubation of bacteria with Peptide 2 renders them susceptible to host clearance. Also, pre-incubation with Peptide 2 markedly increased bacterial killing and phagocytosis in the peritoneal cavity (FIGS. 9D to 9F), consistent with the effect of peptide treatment on phagocytosis and bacterial killing of Kp-1705 infected mice (FIGS. 8A and 8B).

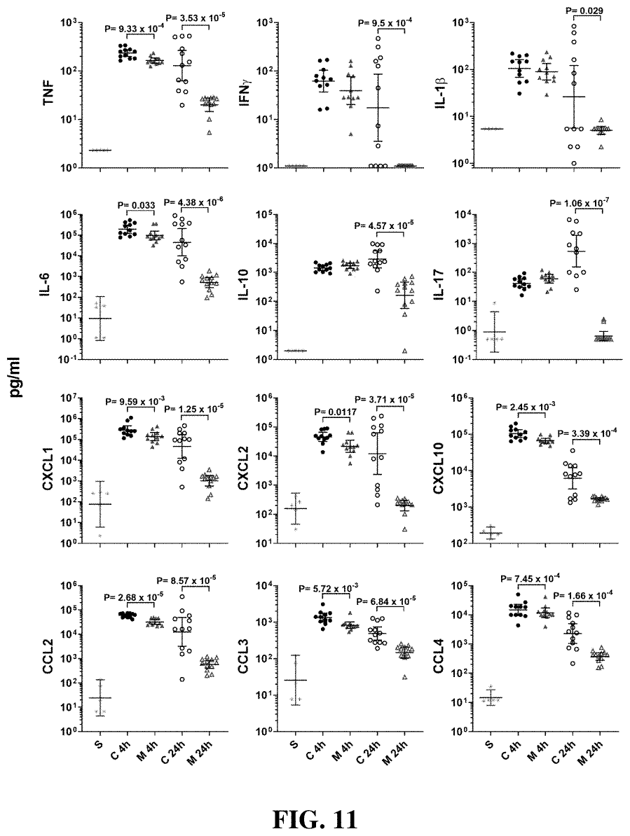

Peptide 2 modulates cytokine responses in Kp-1705 induced sepsis. Inventors analyzed the effect of Peptide 2 treatment on cytokines associated with host responses to infection that are implicated in the immunopathology of bacteremic sepsis. Multiplex analysis of 32 mouse cytokines was performed on plasma samples collected 2, 4, and 24 hours p.i. from Kp-1705 septicemic mice treated with Peptide 2 or saline (FIG. 11 and Table 4). Kp-1705 infection induced a rapid and profound elevation of 26 cytokines, some of which increased >1,000 fold within 2-4 hours p.i. (Table 4). Plasma levels of six cytokines (IL-2, IL-3, IL-4, IL-7 and VEGF) were not significantly altered by Kp-1705 infection nor were they affected by Peptide 2 treatment.

TABLE 4

| | | Sham | Saline | Peptide 1 | | | |
|---|---|---|---|---|---|---|---|
| | Hour | (n = 5) | (n = 11-12) | (n = 11-12) | | % | P- |
| Cytokine | p.I. | Mean ± S.D. | Mean ± S.D. | Mean ± S.D. | Delta | change | Value |
| IL-6 | 2 | 24.5 ± 20.0 | 266274.4 ± 149491.2 | 216618.0 ± 97937.0 | −49656.5 | −18.6 | 0.472 |
| | 4 | | 239682.6 ± 150080.3 | 128253.4 ± 109127.5 | −111429.2 | −46.5 | 0.0328 |
| | 24 | | 224517.4 ± 278673.0 | 687.0 ± 481.4 | −223830.4 | −99.7 | 4.38 × 10⁻⁶ |
| CXCL1 | 2 | 169.7 ± 113.2 | 364564.4 ± 167803.7 | 258876.9 ± 142590.2 | −105687.5 | −29.0 | 0.1260 |
| (KC) | 4 | | 380278.4 ± 305680.2 | 168047.7 ± 111004.5 | −212230.7 | −55.8 | 9.59 × 10⁻³ |
| | 24 | | 118116.7 ± 143173.3 | 1365.9 ± 854.1 | −116750.8 | −98.8 | 1.25 × 10⁻⁵ |
| CXCL10 | 2 | 200.1 ± 58.0 | 29029.4 ± 6754.2 | 37616.2 ± 12095.1 | 8586.9 | 29.6 | 0.126 |
| (IP-10) | 4 | | 110999.9 ± 40362.5 | 8204.6 ± 15429.1 | −42795.3 | −38.6 | 2.45 × 10⁻³ |
| | 24 | | 9666.3 ± 9142.1 | 1667.5 ± 229.7 | −7998.8 | −82.7 | 3.39 × 10⁻⁴ |
| CCL2 | 2 | 48.8 ± 50.7 | 62216.0 ± 7150.9 | 59107.8 ± 9072.5 | −3108.2 | −5.0 | 0.462 |
| (MCP-1) | 4 | | 60693.2 ± 11269.2 | 33486.3 ± 10505.8 | −27206.9 | −44.8 | 2.68 × 10⁻⁵ |
| | 24 | | 53241.7 ± 94318. | 664.5 ± 313.4 | −52577.2 | −98.88 | 8.57 × 10⁻⁵ |
| CCL4 | 2 | 16.3 ± 9.3 | 64892.4 ± 9360.0 | 46231.0 ± 13225.6 | −18661.3 | −28.8 | 9.30 × 10⁻³ |
| (MIP-1β) | 4 | | 35052.8 ± 7096.8 | 24260.1 ± 5045.1 | −10792.7 | −30.8 | 7.45 × 10⁻⁴ |
| | 24 | | 3674.5 ± 4025. | 409.9 ± 163.9 | −3264.6 | −88.8 | 1.66 × 10⁻⁴ |
| CXCL2 | 2 | 208.3 ± 125.0 | 145483.3 ± 23309.5 | 105959.4 ± 38123.4 | −39523.9 | −27.2 | 0.0379 |
| (MIP-2) | 4 | | 51686.3 ± 23415.4 | 27003.7 ± 18839.5 | −24682.6 | −47.8 | 0.0117 |
| | 24 | | 53979.5 ± 70550.0 | 228.1 ± 86.3 | −53751.4 | −99.6 | 3.71 × 10⁻⁵ |
| CCL11 | 4 | 917.8 ± 452.8 | 3962.8 ± 589.1 | 3978.9 ± 435.1 | 16.1 | 0.4 | 0.865 |
| (Eotaxln) | 24 | | 5046.0 ± 2719.7 | 1258.5 ± 296.0 | −3787.5 | −75.1 | 3.24 × 10⁻⁶ |
| IFNy | 4 | 1.1 ± 0.0 | 77.7 ± 48.5 | 57.5 ± 47.9 | −20.2 | −26.0 | 0.246 |
| | 24 | | 109.6 ± 144.9 | 1.1 ± 0.0 | −108.5 | −99.0 | 9.45 × 10−⁴ |
| IL-1α | 4 | 67.5 ± 112.1 | 277.9 ± 80.6 | 282.8 ± 103.6 | 4.9 | 1.8 | 0.973 |
| | 24 | | 1030.9 ± 1915.3 | 93.7 ± 57.8 | −937.2 | −90.9 | 4.89 × 10⁻³ |
| IL-1β | 4 | 5.4 ± 0.0 | 123.7 ± 62.2 | 104.9 ± 60.0 | −18.8 | −15.2 | 0.548 |
| | 24 | | 177.9 ± 267.3 | 5.2 ± 1.3 | −172.6 | −97.1 | 0.0288 |
| IL-5 | 4 | 1.0 ± 0.0 | 154.6 ± 131.5 | 189.3 ± 138.1 | 34.6 | 22.4 | 0.294 |
| | 24 | | 485.2 ± 629.9 | 16.2 ± 21.2 | −469.0 | −96.7 | 7.07 × 10⁻⁴ |
| IL-9 | 4 | 126.5 ± 197.2 | 236.4 ± 139.2 | 491.8 ± 298.9 | 255.4 | 108.0 | 0.032 |
| | 24 | | 695.5 ± 709.8 | 49.9 ± 54.1 | −645.5 | −92.8 | 7.30 × 10⁻³ |
| IL-10 | 4 | 2.0 ± 0.0 | 1472.0 ± 417.1 | 1768.1 ± 505.6 | 296.1 | 20.1 | 0.196 |
| | 24 | | 4275.1 ± 3340.0 | 296.6 ± 216.3 | −3978.5 | −93.1 | 4.57 × 10⁻⁴ |
| IL-12 | 4 | 3.9 ± 0.0 | 166.0 ± 79.8 | 137.7 ± 34.3 | −28.3 | −17.0 | 0.504 |
| (p40) | 24 | | 25.6 ± 23.7 | 3.9 ± 0.0 | −21.7 | −84.7 | 3.78 × 10⁻³ |
| IL-12 | 4 | 4.8 ± 0.0 | 180.8 ± 42.1 | 200.0 ± 67.6 | 19.1 | 10.6 | 0.650 |
| (p70) | 24 | | 129.4 ± 110.4 | 4.8 ± 0.0 | −124.6 | −96.3 | 7.50 × 10⁻⁴ |
| LIF | 4 | 1.0 ± 0.0 | 37.1 ± 15.8 | 28.6 ± 22.8 | −8.5 | −22.9 | 0.108 |
| | 24 | | 514.1 ± 677.1 | 1.0 ± 0.0 | −513.1 | −99.8 | 1.59 × 10⁻⁶ |
| IL-13 | 4 | 103.7 ± 23.8 | 276.2 ± 54.4 | 311.0 ± 71.2 | 34.8 | 12.6 | 0.302 |
| | 24 | | 211.9 ± 118.2 | 25.7 ± 16.0 | −186.2 | −87.9 | 7.21 × 10 − 7 |
| CXCL5 | 4 | 1581.1 ± 715.6 | 2830.2 ± 1294.5 | 3115.6 ± 2014.5 | 285.4 | 10.1 | 0.981 |
| (LIX) | 24 | | 4240.1 ± 2475.4 | 678.2 ± 303.6 | −3561.9 | −84.0 | 2.62 × 10 − 6 |
| IL-15 | 4 | 124.9 ± 92.8 | 382.9 ± 161.3 | 642.5 ± 276.2 | 259.7 | 67.8 | 9.14 × 10⁻³ |
| | 24 | | 662.1 ± 335.2 | 391.3 ± 355.3 | −270.9 | −40.9 | 0.0519 |
| IL-17 | 4 | 2.2 ± 3.4 | 46.4 ± 21.5 | 66.4 ± 27.4 | 19.9 | 42.9 | 0.111 |
| | 24 | | 1613.4 ± 2215.2 | 0.8 ± 0.7 | −1612.6 | −100.0 | 1.06 × 10⁻⁷ |
| CCL3 | 4 | 46.1 ± 44.1 | 1446.7 ± 625.9 | 866.2 ± 318.7 | −580.5 | −40.1 | 5.72 × 10⁻³ |
| (MIP-1α) | 24 | | 595.6 ± 381.8 | 161.8 ± 58.9 | −433.8 | −72.8 | 6.84 × 10⁻⁵ |
| M-CSF | 4 | 3.5 ± 0.0 | 122.8 ± 60.3 | 118.1 ± 50.1 | −4.8 | −3.9 | −0.947 |
| | 24 | | 175.4 ± 219.1 | 7.8 ± 6.4 | −167.6 | −95.5 | 8.88 × 10⁻³ |
| GM- | 4 | 10.9 ± 0.0 | 84.5 ± 16.2 | 75.2 ± 24.8 | −9.2 | −10.9 | 0.282 |
| CSF | 24 | | 102.9 ± 109.8 | 10.9 ± 0.0 | −92.0 | −89.4 | 1.15 × 10⁻³ |
| CXCL9 | 4 | 34.8 ± 29.1 | 4108.2 ± 476.4 | 3811.4 ± 778.2 | −296.8 | −7.2 | 0.265 |
| (MIG) | 24 | | 4839.0 ± 3354.1 | 873.1 ± 208.2 | −3965.9 | −82.0 | 1.61 × 10⁻⁵ |
| CCL5 | 4 | 3.9 ± 2.4 | 1205.2 ± 314.6 | 722.8 ± 390.1 | −482.4 | −40.0 | 0.0166 |
| (RANTES) | 24 | | 1003.9 ± 680.1 | 118.3 ± 42.0 | −885.6 | −88.2 | 6.57 × 10⁻⁶ |
| TNF | 4 | 2.3 ± 0.0 | 241.2 ± 56.7 | 166.6 ± 30.3 | −74.6 | −30.9 | 9.33 × 10⁻⁴ |
| | 24 | | 214.3 ± 190.9 | 21.9 ± 7.0 | −192.4 | −89.8 | 3.53 × 10⁻⁵ |

Table 4 summarizes results of studies in which mice were challenged i.p. with Kp-1705 and treated 1 hours later with Peptide 2 (1.25 mg/kg i.p.) or saline. The sham cohort received bacteria-free suspension buffer at t=0 followed by saline 1 hours later. Mice were euthanized 2, 4, or 24 hours post infection, and plasma samples were subjected to multiplex cytokine analysis. P values calculated using ANOVA with Uncorrected Fisher's LSD comparing saline and Peptide 2 treated mice at respective time points.

Peptide 2 treatment had selective and differential effects on initial early (2 and 4 hours p.i.) and later (24 hours p.i.) cytokine responses in Kp-1705 infected mice (FIG. 11 and Table 4). The earliest (2 hours) effects seen were statistically significant reductions of CCL4 and CXCL2 (Table 4). By 4 hours p.i., 7 additional cytokines were significantly reduced, including TNF, IL-6, and CXCL1 (KC) which have major roles in sepsis pathogenesis (FIG. 11). Peptide 2 treatment also resulted in significant increases of IL-9 and IL-15 levels at 4 hours p.i. (Table 4).

Although several early (2 or 4 hours) cytokine responses were selectively regulated by Peptide 2 treatment, there was a global reduction in all 26 plasma cytokines affected by Kp-1705 infection at the 24 hour time point, including those that had been increased initially (IL-9 and IL-15; FIG. 11 and Table 4). With the exception of IL-15 (P=0.052), reductions of cytokine levels were significant compared to saline treated controls (Table 4), and levels of 23 cytokines were reduced by >80%, most notably TNF, (−89.8%; P=3.52×10^{-s}), IL-6 (−99.7%; P=4.38×10⁻⁶), CXCL1 (KC) (−98.8%; P=1.25×10⁻⁵), and IL-17 (−100.0%; P=1.06×10⁻⁷) (FIG. 11). Moreover, Peptide 2 treatment restored 14 cytokines to sham control levels by 24 hours (FIG. 11, Table 4).

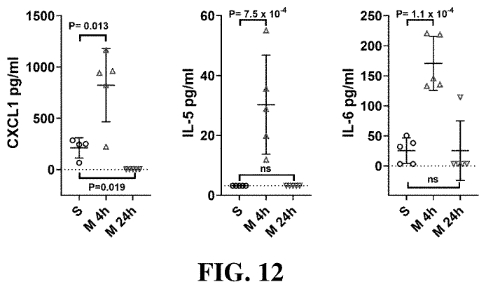

The effect of Peptide 2 administration on plasma cytokines was also analyzed in uninfected mice that received saline (sham infection controls) followed 1 hour later with a single i.p. dose of 1.25 mg/kg peptide. At the 4 hour collection point, only three of the 32 mouse cytokines changed significantly relative to sham/saline controls: IL-5, IL-6, and CXCL1 were significantly elevated compared to vehicle treated controls (FIG. 12). However, all three cytokines returned to baseline by 24 hours.

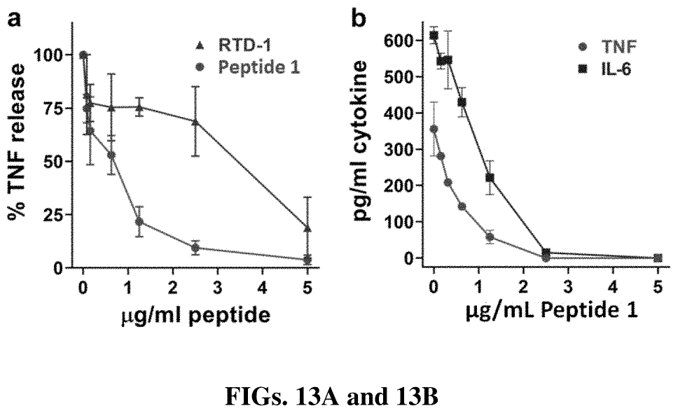

Peptide 2 modulates cytokine responses in vitro. The effects of Peptide 2 and RTD-1 on cytokine responses was further analyzed by incubating LPS-stimulated mouse RAW 264.7 macrophages with 0-5 μg/ml Peptide 2 or RTD-1. RTD-1 suppressed the release of soluble TNF from LPS stimulated macrophages dose dependently (FIG. 13A). Peptide 2 also inhibited LPS-stimulated TNF release, but 50% TNF suppression was obtained at ca. 4-fold lower concentration than RTD-1.

The effect of Peptide 2 on cytokines released by human whole blood was characterized by incubation with live Kp-1705 cells. As shown in FIG. 13B, Peptide 2 concentration-dependently suppressed the release of TNF and IL-6. Peptide alone had no effect on TNF or IL-6 release (data not shown). These results indicate that the effects of Peptide 2 in modulating cytokine responses in vivo in mice and in mouse RAW 264.7 macrophages extend to TNF and IL-6 release by human blood leukocytes.

Peptide 2 stability. Preliminary studies of Peptide 2 indicated that the peptide is exceedingly stable in aqueous media including acidic solutions as low as pH 2.0. To assess peptide stability further, Peptide 2 was incubated in human EDTA-plasma (90% vol/vol) at 37° C. for 48 hours. Reversed-phase UPLC analyses showed that >95% of the peptide remained intact. In addition, 1.25 μg/ml Peptide 2 was incubated with log-phase Kp-1705 or E. coli ATCC-BAA-2340 suspended in 50 mM HEPES at 37° C. for 24 hours. Intact Peptide 2 was quantified by LC-MS/MS which showed 72.4±0.93% and 81.3±2.1% of Peptide 2 was found intact post Kp-1705 and E. coli incubations respectively. These findings demonstrate that Peptide 2 is remarkably resistant to bacterial proteases.

### Materials and Methods

All methods were performed in accordance with relevant federal, state, and institutional guidelines. Animal use protocols were approved by The University of Southern California (USC) Institutional Animal Care and Use Committee (IACUC), Protocol #20538. Blood was obtained from healthy adult volunteers according to approved USC Institutional Review Board (IRB) Protocol HS-09-00280.

Peptides and antibiotics. The hydrochloride salts of Peptide 2 and RTD-1 (>95%) were produced by solid phase peptide synthesis as described 10,14. Imipenem (I0160), meropenem (M2574), ceftazidime (A6987), colistin (C4461), and polymyxin B (P0972) were purchased from Sigma-Aldrich (St. Louis, Mo.). Stock solutions of Peptide 2, RTD-1, and antibacterial drugs were prepared as concentrated stocks in sterile water. For animal injection, peptides and antibacterial drugs were diluted to the indicated concentrations in sterile normal saline.

Bacterial strains. Bacterial strains were obtained from American Type Culture Collection. K. pneumoniae strains included BAA-1705 (blaKPC-2, hypermucoid), BAA-2146 (blaNDM-1), BAA-700603 (SHV-18, extended spectrum β-lactamase, ESBL), and BAA-1706 (non-CRE reference strain). E. coli strains included BAA-2340 (blaKPC-2), BAA-2471 (blaNDM-1), and ML35 (non-CRE reference strain).

Antibacterial assays. Minimum inhibitory concentration (MIC) assays were performed using Clinical and Laboratory Standards Institute (CLSI; document M27-A2) protocols. Bacteria were incubated with antibiotics or peptide for 20 hours at 37° C., and bacterial growth was determined by A₆₂₀ using a SpectraMax® M5e plate reader. MIC was the lowest agent concentration that completely inhibited growth as determined by A₆₂₀ absorbance.

K. pneumoniae and E. coli bacteremia. BALB/c mice (Jackson Laboratories) 8-10 weeks of age were acclimated (3-5 to a cage for at least 5 days prior to infection) in a thermostatically controlled room with 12 hours light/dark cycle. Cryopreserved K. pneumoniae 47 cells were thawed and adjusted to a density of 6-10×10⁸ CFU/ml, confirmed by plating on trypticase soy agar (TSA) plates. E. coli BAA-2340 was grown to log-phase in trypticase soy broth, pelleted, washed with PBS, and resuspended in PBS at 0.5-1×10⁹ CFU/ml. Mice were infected by i.p. injection of 2-5×10⁸ CFU of Kp-1705 or 3-6×10⁷ CFU of E. coli BAA-2340 using a 28 g needle in a volume of 0.5 ml. Post-infection, mice were observed three times a day for at least 4 days and then daily for 28 days. Mice were euthanized when they became moribund. All experiments used both male and female mice and were repeated at least twice.

Bacterial burden determination. Blood, peritoneal lavage fluid, and spleen homogenates were obtained from mice following by CO₂ euthanasia. Citrate-anti-coagulated blood was collected by aseptic cardiac puncture. Peritoneal lavage was performed by injecting 3 ml of PBS into the peritoneal cavity, massaging the abdomen, and then collecting fluid aseptically through a minor incision in the abdominal wall. Spleen was surgically removed and homogenized in sterile PBS using Polytron® PT10-35 homogenizer. Samples were serially diluted and plated in triplicate on TSA plates, incubated overnight at 37° C. and colonies counted.

Hematology and cytokine analyses. EDTA-anticoagulated blood was collected aseptically by terminal cardiac puncture and analyzed for complete blood cell count using an Element® HT5 hematology analyzer (Heska). Blood plasma and clarified peritoneal lavage were prepared by two-step centrifugation described previously²⁴. Plasma cytokine levels were quantified using a mouse-specific MILLIPLEX MAP kit (Millipore Cat #MCYTMAG-70K) as described 11.

Peptide 2 pharmacokinetics. Single dose PK of Peptide 2 was evaluated by quantifying plasma peptide concentrations following 1.25 mg/kg injections administered i.p. to 2M/2F BALB/c mice. EDTA plasma was prepared from blood collected aseptically by terminal cardiac puncture. Peptide 2 plasma concentrations were determined by reverse-phase liquid chromatography (XBridge® phenyl 3.5 μm column, Waters) on an Acquity® H-Class UPLC (Waters) with tandem electrospray mass spectroscopy on a Xevo® TQ-S running MassLynx® V4.1 (Waters).

Cell culture. Mouse RAW 264.7 macrophages (RAW cells; ATCC, TIB-71) were grown in Dulbecco's Modified Eagle Medium (DMEM) with 10% heat-inactivated fetal bovine serum (HI-FBS) with 100 U/ml penicillin/streptomycin, and suspended to 2-3×10⁶ cells/ml in fresh media before use.

Phagocytosis assay. Kp-1705 was grown overnight, harvested by centrifugation, washed twice, resuspended in Hank's Balanced Salt Solution (HBSS), and adjusted to an OD A₆₂₀=0.5 (2.5×10⁸ CFU/ml). Bacteria were pre-incubated with varied concentrations of Peptide 2 at room temperature for 30 minutes, and incubated (MOI 50:1) with RAW cells with orbital shaking at 37° C. for 30 minutes. Cytospin® preparations of 100 μl samples were then prepared, fixed with methanol, and stained with hematoxylin and eosin (H & E). Slides were viewed at 100× and 50 RAW cells selected from random fields were manually counted for number of cell associated bacteria. All samples were run in duplicate and experiments performed twice.

LPS stimulation of RAW cells. RAW cells were seeded into 48-well tissue culture plates at 1-1.5×10⁵ cells/well and allowed to adhere overnight. Cells were washed twice and suspended in DMEM with 1% FBS with 0-5 μg/ml of Peptide 2 immediately followed by addition of 5 ng/ml of E. coli 0111:B4 LPS (Sigma L4391). Plates were incubated for 2 hours at 37° C. in 5% CO₂. Well contents were removed, clarified by centrifugation, and supernatants frozen at −80° C. Supernatant TNF was quantified by mouse TNF ELISA (Invitrogen, Cat #BMS607-3).

Bacterial stimulation of human blood. Log-phase Kp-1750 grown in TSB were pelleted, washed twice in PBS, and suspended to 1×10⁵ CFU/ml in RPMI-1640. EDTA-anticoagulated blood collected from a healthy adult human donor was incubated at a final dilution of 1:10 in RPMI-1640 with 1000 CFU/ml of Kp-1705 and varied concentrations of Peptide 2 for 4 hours at 37° C. in 5% CO₂ with gentle rocking. Incubation supernatants were frozen at −80° C. TNF and IL-6 were quantified by ELISA (Invitrogen KHC3011, KHC0061).

Peptide stability analysis. Peptide 2 (final concentration of 100 μg/ml) was added to 90% human EDTA plasma and incubated at 37° C. for 48 hours. Samples were processed by addition of 10% HOAc and 5% ACN (final concentrations) at time 0 and 48 hours, and Peptide 2 was quantified by C18 RP-HLPC on an Acquity H-Class UPLC with an analytical PDA detector using Empower 3 software (Waters). All samples were performed with technical replicates and analyzed by UPLC in duplicate and the experiment repeated once. In separate experiments, Peptide 2 (1.25 μg/ml final) was incubated with log phase Kp-1705 (5×10⁷ CFU/ml) in 50 mM HEPES pH 7.4. A time 0 hour sample was collected, and the remaining suspension incubated at 37° C. for 24 hours. Samples were processed by addition of 5% formic acid/5% acetonitrile, vortexed, and clarified at 22,000×g for 10 minutes. Supernatant Peptide 2 was then quantified by LC-MS/MS as described in PK section above. All samples were performed with technical replicates and analyzed by LC-MS/MS in duplicate.

In vivo phagocytosis and peritoneal bioburden determination. Cryopreserved Kp-1705 was suspended in PBS to a density of ca. 1×10⁹ CFU/ml, and incubated with saline or 1.25 μg/ml Peptide 2 at room temperature for 1 hour. Mice (n=26) were then infected i.p. with 500 μl of saline—or Peptide 2—pretreated Kp-1705, and a third cohort (n=12) received 500 μl i.p. of 1.25 μg/ml Peptide 2 immediately following infection with saline treated Kp-1705. Five mice from each cohort were euthanized 4 hours p.i., and peritoneal lavage was collected for bacterial burden determination and Cytospin® preparation. In vitro antibacterial activity of Peptide 2 (1.25 ug/ml)-treated cells (1 h; 37° C.) was determined by plating saline or peptide treated cells on TSA plates. Growth kinetics of saline and Peptide 2-treated cells was determined by diluting samples from the 1 hour incubation mixtures 1:40 into TSB and monitoring growth (620 nm) for 5.5 hours at 37° C. Antibacterial activity of Peptide 2 in PF was determined by i.p. injection of naïve male BALB/c mice (n=5 per group) with 1.25 mg/kg of Peptide 2 or saline i.p. (10 ml/kg). Mice were euthanized 1 hour after i.p. injection and peritoneal fluid was harvested aseptically using a micropipette introduced into a surgical incision in the abdominal wall. Peritoneal fluid was clarified by centrifugation at 1,200×g and an aliquot used to quantify Peptide 2 by LC-MS/MS, as described above. Antimicrobial activity was determined by incubating PF (9:1 vol/vol) with Kp-1705 at a final density of 3×10⁷ CFU/ml. Samples were incubated at 37° C. for 1 hour, serially diluted, and plated on TSB to determine bacterial viability. Statistical Analyses. All statistical analyses were performed using GraphPad Prism® Version 8.2.1. End-point survival data were compared using Fisher's exact test. P values of ≤0.05 were defined as significant. Statistics analyzing bacterial burdens and cytokine responses first confirmed lognormal distribution by Anderson-Darling test. Values were then log-transformed and analyzed by one-way ANOVA with an Uncorrected Fisher's Least Significant Difference (LSD) post-test. Student's t-tests were performed using a two-tail analysis.

Analogs of θ-defensins as described herein can be applied using any suitable method. For example, such analogs can be provided by injection or infusion. The high degree of effectiveness observed for some θ-defensin analogs indicates that these can be provided to an individual in need of treatment in effective amounts by simple subcutaneous, intradermal, subdermal, intravenous, and/or intramuscular injection.

Alternatively, the low molecular weight and high degree of stability conferred by circular structure and the presence of disulfide bonds can allow for oral administration of θ-defensin analogs of the inventive concept. Such oral administration can include administration of a solution or suspension of the θ-defensin analog in a liquid pharmaceutical carrier suitable for oral administration. In some embodiments a θ-defensin analog can be provided in a dry or lyophilized form that is reconstitute in a liquid media prior to oral administration. Such dry or lyophilized formulations can include a stabilizer. Suitable stabilizers include carbohydrates (e.g., mannitol, sucrose, trehalose) and/or proteins (e.g., albumin).

Alternatively, analogs of θ-defensin can be provided in a tablet, capsule, pill, or other suitable solid and compact form for oral administration. Such formulations can include coatings, shells, or similar components that provide for delayed release of the θ-defensin analog (for example, delaying release until reaching the small intestine). Such formulations can include the θ-defensin in liquid form within an enclosure or coating. Alternatively, such formulations can include a θ-defensin analog in a dry or lyophilized form. Suitable dry or lyophilized forms include powders, granules, and compressed solids. Such dry or lyophilized formulations can include a stabilizer. Suitable stabilizers include carbohydrates (e.g., mannitol, sucrose, trehalose) and/or proteins (e.g., albumin).

As noted above, θ-defensin analogs of the inventive concept can effectively treat sepsis and/or septic shock. In some embodiments such treatment is in response to an ongoing, acute condition. In other embodiments such treatment is prophylactic, for example used to prevent the development of septic shock when the individual is suspected of having sepsis or a high probability of developing sepsis. Treatment can be provided by administration of a θ-defensin analog of the inventive concept on any suitable schedule. For example, a θ-defensin analog can be provided as a single dose, periodic doses, or as a continuous infusion. Periodic doses can be administered at any suitable intervals. Suitable intervals can be hourly, every 2 hours, every 4 hours, 4 times a day, 3 times a day, twice a day, once daily, every 2 days, every 3 days, twice a week, weekly, every 2 weeks, every 4 weeks, every 2 months, every 3 months, every 4 months, 3 times a year, twice a year, or annually.

In some embodiments the mode of administration for a θ-defensin analog can be modified during the course of treatment. For example, a θ-defensin analog of the inventive concept can initially be administered by intravenous injection or infusion (e.g., to rapidly provide effective concentrations in acute sepsis or septic shock), followed by intradermal injection, intramuscular injection, and/or oral administration in order to maintain an effective concentration over a remaining period of treatment.

For prophylactic use, a θ-defensin analog can be administered prior to the onset of observable symptoms. For treatment of an active disease or condition a θ-defensin analog can be administered for a period of suitable to effectively treat the disease or condition. Such a period can be over for a controlled period of time, or can be long term (e.g., for treatment of chronic conditions).

In some embodiments of the inventive concept a θ-defensin analog can be used in combination with other pharmaceutically active compounds. Suitable compounds include a θ-defensin, a different θ-defensin analog, an antibacterial antibiotic, an antiviral, an antifungal antibiotic, an anti-inflammatory drug (e.g., steroids, non-steroidal anti-inflammatory drugs), a vasopressor, and/or a biologic (e.g., antibodies or antibody fragments). Such additional pharmaceutical compounds can be provided on the same schedule as the θ-defensin analog, or on an independent schedule. In some embodiments a θ-defensin analog-containing formulation can be provided that incorporates one or more of such additional pharmaceutically active compounds. Inventors believe that such cotherapy can provide a synergistic effect in which the cumulative effect of administration of the θ-defensin analog in combination with the additional pharmaceutically active compound exceeds the sum of the individual effects observed with treatment using the θ-defensin analog and the additional pharmaceutically active compound in amounts corresponding to those used for cotherapy.

It is contemplated that the host defenses modulated by θ-defensins and/or θ-defensin analogs can provide an antiviral and/or antifungal effect via modulation of host defense mechanisms, and can prove useful in treating viral and/or fungal disease in amounts that do not provide a direct antiviral or antifungal activity—particularly in species or strains that are resistant to conventional antiviral and/or antifungal compounds. Such treatment includes prophylaxis and/or active disease. In some embodiments active disease so treated is symptomatic. In other embodiments active disease so treated is asymptomatic.

Inventors believe that θ-defensin analogs as described herein can be effective at treating a variety of conditions resulting from dysregulation of the immune or inflammatory response, including chronic conditions. Examples of such chronic conditions include rheumatoid arthritis and inflammatory bowel disease.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A method of treating an infection in a subject in need thereof with a microbe, comprising administering a small peptide in an amount effective to modulate two or more of microbial clearance, phagocytosis, neutrophil recruitment, and septic shock, wherein the amount does not provide a direct antimicrobial effect.

2. The method of claim 1, wherein the small peptide is a macrocyclic peptide, theta defensin or a theta defensin analog.

3. The method of claim 1, wherein the microbe is resistant to one or more antibiotic(s).

4. The method of claim 1, wherein modulation comprises enhancement of microbial clearance.

5. The method of claim 1, wherein modulation comprises enhancement of phagocytosis of the microbe.

6. The method of claim 1, wherein modulation comprises an increase in neutrophil recruitment.

7. The method of claim 1, wherein modulation comprises reduction or increase in a pro-inflammatory cytokine.

8. The method of claim 1, wherein modulation comprises increase or reduction in an anti-inflammatory cytokine.

9. The method of claim 1, wherein the small peptide is selected to be resistant to a microbial protease.

10. The method of claim 1, wherein the small peptide is administered in an amount effective to modulate three or more of microbial clearance, phagocytosis, neutrophil recruitment, and septic shock, wherein the amount does not provide a direct antimicrobial effect.

11. The method of claim 1, wherein the small peptide has the following structure: 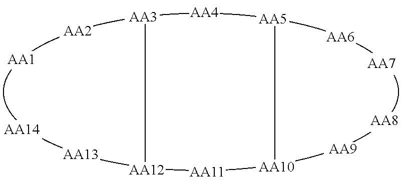
wherein AA3 and AA12 are cysteines joined by a disulfide bond, AA5 and AA10 are cysteines joined by a disulfide bond, AA4 is a first hydrophobic amino acid, AA11 is a second hydrophobic amino acid, AA6 is arginine, AA7 is arginine, AA8 is arginine, and wherein the cyclic peptide has four arginine residues that provide a positively charged content of about 28% at physiological pH.

12. The method of claim 1, wherein the small peptide has the following structure: 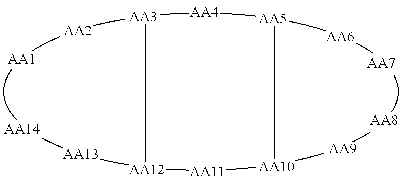
wherein AA3 and AA12 are cysteines joined by a disulfide bond, AA5 and AA10 are cysteines joined by a disulfide bond, AA4 is serine or a first hydrophobic amino acid, AA11 is serine or a second hydrophobic amino acid, AA6 is arginine, AA7 is arginine, AA8 is arginine, and wherein the cyclic peptide comprises five arginine residues that provide a positively charged content of at least about 36% at physiological pH.

13. A composition for treating microbial infection, comprising:
a small peptide selected to be effective in modulating two or more of microbial clearance, phagocytosis, neutrophil recruitment, and septic shock in an amount does not provide a direct bactericidal effect; and
a compound selected from the group consisting of an antibacterial antibiotic, an antiviral, an antifungal antibiotic, an anti-inflammatory drug, a vasopressor, and an antibody or antibody fragment.

14. The composition of claim 13, wherein the small peptide is macrocyclic peptide, a theta defensin or a theta defensin analog.

15. The composition of claim 13, wherein the microbe is resistant to an antibiotic.

16. The composition of claim 13, wherein the microbe is resistant to a plurality of antibiotics.

17. The composition of claim 13, wherein the small peptide is selected to be resistant to a microbial protease.

18. The composition of claim 13, wherein the small peptide has the following structure: 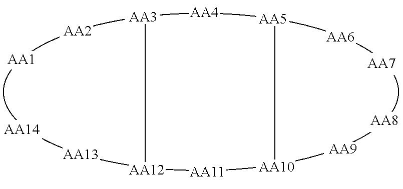
wherein AA3 and AA12 are cysteines joined by a disulfide bond, AA5 and AA10 are cysteines joined by a disulfide bond, AA4 is a first hydrophobic amino acid, AA11 is a second hydrophobic amino acid, AA6 is arginine, AA7 is arginine, AA8 is arginine, and wherein the cyclic peptide has four arginine residues that provide a positively charged content of about 28% at physiological pH.

19. The composition of claim 13, wherein the small peptide has the following structure: 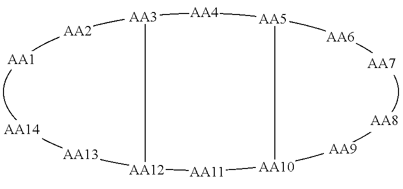
wherein AA3 and AA12 are cysteines joined by a disulfide bond, AA5 and AA10 are cysteines joined by a disulfide bond, AA4 is serine or a first hydrophobic amino acid, AA11 is serine or a second hydrophobic amino acid, AA6 is arginine, AA7 is arginine, AA8 is arginine, and wherein the cyclic peptide comprises five arginine residues that provide a positively charged content of at least about 36% at physiological pH.
